(19) United States
(12) Patent Application Publication
(10) Pub. No.: US 2025/0140365 A1
(43) Pub. Date: May 1, 2025

(54) SUPPORT APPARATUS, SUPPORT METHOD, AND NON-TRANSITORY COMPUTER READABLE MEDIUM STORING PROGRAM

(71) Applicant: NEC Corporation, Minato-ku, Tokyo (JP)

(72) Inventors: Yuki KOSAKA, Tokyo (JP); Shigemi Kitahara, Tokyo (JP)

(73) Assignee: NEC Corporation, Minato-ku, Tokyo (JP)

(21) Appl. No.: 18/692,369

(22) PCT Filed: Sep. 28, 2022
(86) PCT No.: PCT/JP2022/036148

§ 371 Date: Mar. 15, 2024

(30) Foreign Application Priority Data
Oct. 11, 2021 (JP) 2021-166976

(51) Int. Cl. G16H 20/30; G06F 16/31; G08B 5/36

(52) U.S. Cl. CPC G16H 20/30; G06F 16/313; G08B 5/36

(57) ABSTRACT

A support apparatus capable of efficiently checking whether or not a patient problem is appropriately set is provided. A support apparatus includes a prediction unit, a calculation unit, and an output unit. The prediction unit predicts a patient problem to be dealt with in order to achieve a target of the patient. The calculation unit calculates a matching level between a predicted patient problem and an actual patient problem, the predicted patient problem being a predicted problem of the patient, and the actual patient problem being a problem of the patient that has been actually dealt with for the patient. The output unit performs control so that an alert is output when the calculated matching level is lower than a predetermined first threshold.

### TECHNICAL FIELD

The present invention relates to a support apparatus, a support method, and a non-transitory computer readable medium storing a program.

### BACKGROUND ART

In facilities (rehabilitation hospitals and the like) where activities aimed at improving abilities including physical functions, such as rehabilitation, (hereinafter referred to simply as “activities”) are carried out, therapists such as physical therapists often conduct treatments, such as by helping patients or the like to carry out rehabilitation, under the direction of doctors. In this regard, Patent Literature 1 discloses a medical information display device that supports the updating of a rehabilitation plan in which daily conditions of a patient are taken into consideration. The apparatus disclosed in Patent Literature 1 acquires rehabilitation information indicating conditions of the patient when he/she is carrying out rehabilitation and daily information indicating conditions of the patient when he/she is not carrying out rehabilitation. Further, the apparatus disclosed in Patent Literature 1 outputs a rehabilitation screen in which the rehabilitation information and the daily information are displayed in a temporally-synchronized manner.

### CITATION LIST

### Patent Literature

Patent Literature 1: Japanese Unexamined Patent Application Publication No. 2020-184219

### SUMMARY OF INVENTION

### Technical Problem

When a therapist conducts a treatment on a patient, such as by helping a patient to carry out rehabilitation, (hereinafter also referred to simply as “treatment”), the therapist often determines, for example, the contents of the rehabilitation after setting a problem(s) of the patient to be solved under the direction of a doctor in order to achieve a target of activities of the patient. Note that if the problem of the patient (hereinafter also referred to as the patient problem) is not appropriately set, a treatment(s) necessary to achieve the target cannot be conducted, so that it may not be possible to achieve the target. Therefore, it is necessary to check whether or not the patient problem is appropriately set.

The present disclosure has been made to solve the above-described problem, and an object thereof is to provide a support apparatus, a support method, and a program capable of efficiently checking whether or not a patient problem is appropriately set.

### Solution to Problem

A support apparatus according to the present disclosure includes: prediction means for predicting a patient problem to be dealt with in order to achieve a target of the patient; calculation means for calculating a matching level (concordance level) between an actual patient problem and a predicted patient problem, the actual patient problem being a problem of the patient that has been actually dealt with for the patient, and the predicted patient problem being a predicted problem of the patient; and output means for performing control so that an alert is output when the matching level is lower than a predetermined first threshold.

Further, a support apparatus according to the present disclosure includes: predicting a patient problem to be dealt with in order to achieve a target of the patient; calculating a matching level between an actual patient problem and a predicted patient problem, the actual patient problem being a problem of the patient that has been actually dealt with for the patient, and the predicted patient problem being a predicted problem of the patient; and performing control so that an alert is output when the matching level is lower than a predetermined first threshold.

Further, a program according to the present disclosure causes a computer to perform: a step of predicting a patient problem to be dealt with in order to achieve a target of the patient; a step of calculating a matching level between an actual patient problem and a predicted patient problem, the actual patient problem being a problem of the patient that has been actually dealt with for the patient, and the predicted patient problem being a predicted problem of the patient; and a step of performing control so that an alert is output when the matching level is lower than a predetermined first threshold.

### Advantageous Effects of Invention

According to the present disclosure, it is possible to provide a support apparatus, a support method, and a program capable of efficiently checking whether or not a patient problem is appropriately set.

### BRIEF DESCRIPTION OF DRAWINGS

FIG. 1 shows an outline of a support apparatus according to an example embodiment of the present disclosure;

FIG. 2 is a flowchart showing a support method performed by a support apparatus according to an example embodiment of the present disclosure;

FIG. 3 shows a support system according to a first example embodiment;

FIG. 4 shows a configuration of a support apparatus according to the first example embodiment;

FIG. 5 is a diagram for explaining timings at which processes are performed by a support apparatus according to the first example embodiment;

FIG. 6 shows an example of a predicted patient problem and an actual patient problem according to the first example embodiment;

FIG. 7 shows an example of patient information according to the first example embodiment;

FIG. 8 is a flowchart showing a support method performed by a support apparatus according to the first example embodiment;

FIG. 9 is a flowchart showing a first example of a method for calculating a prediction matching level performed by a prediction matching level calculation unit according to the first example embodiment;

FIG. 10 is a flowchart showing a second example of a method for calculating a prediction matching level performed by a prediction matching level calculation unit according to the first example embodiment;

FIG. 11 is a flowchart showing a method for determining a type of an alert issued by an alert type determination unit according to the first example embodiment;

FIG. 12 shows examples of alerts output under the control of an alert output unit according to the first example embodiment;

FIG. 13 is a flowchart showing a support method performed by a support apparatus according to a second example embodiment; and

FIG. 14 shows examples of alerts that are output under control of an alert output unit according to the second example embodiment.

### EXAMPLE EMBODIMENT

### Outline of Example Embodiment According to Present Disclosure

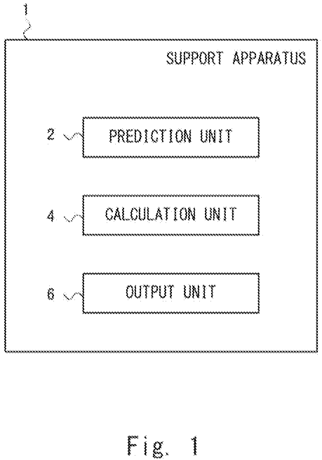

Prior to describing an example embodiment according to the present disclosure, an outline of the example embodiment according to the present disclosure will be described. FIG. 1 shows an outline of a support apparatus 1 according to an example embodiment of the present disclosure. The support apparatus 1 is, for example, a computer such as a server or a personal computer.

The support apparatus 1 performs processing related to the setting of a problem (patient problem) to be solved in order to achieve a target of an activity(ies) (ability improving activity(ies)) of a patient aimed at improving his/her ability(ies), such as the target of rehabilitation. Note that in the following description, an example where the ability improving activity is a treatment such as rehabilitation will be described, but the ability improving activity is not limited to rehabilitation (treatment).

The support apparatus 1 includes a prediction unit 2, a calculation unit 4, and an output unit 6. The prediction unit 2 has a function as prediction means. The calculation unit 4 has a function as calculation means. The output unit 6 has a function as output means.

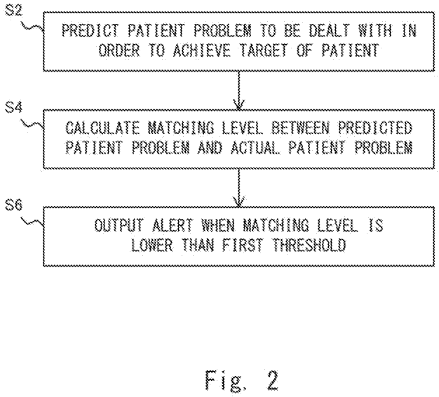

FIG. 2 is a flowchart showing a support method performed by the support apparatus 1 according to the example embodiment of the present disclosure. The prediction unit 2 predicts a patient problem to be dealt with in order to achieve the target of the patient (step S2). The calculation unit 4 calculates a matching level between a predicted patient problem, which is a predicted problem of the patient, and an actual patient problem, which is a problem of the patient that has actually been dealt with for the patient (Step S4). The output unit 6 performs control so that an alert is output when the calculated matching level is lower than a predetermined first threshold (Step S6).

Note that the output alert corresponds to a warning indicating that the target may not be achieved because the patient problem may not be appropriately set. Further, the output unit 6 may perform control so as to display an alert on a display device (user interface) provided in the support apparatus 1. Alternatively, the output unit 6 may perform control so as to display an alert on an apparatus different from the support apparatus 1, such as a user terminal. Further, the output unit 6 may perform control so as to output an alert by means of a sound, a voice, or the like.

For example, in a rehabilitation hospital, in order to optimize a hospitalization period, a time of discharge (hereinafter also referred to as a discharge time) and a target condition(s) of the patient at the time of discharge (hereinafter simply referred to as a “target”) is set, and rehabilitation is planned and carried out for achieving the target. However, the target may not be achieved as being planned, and a “delay in discharge” (hereinafter also referred to as a discharge delay) may occur, i.e., the patient may not be able to be discharged at the set discharge time. Therefore, it is desired to prevent or reduce the discharge delay.

One of the causes of such a discharge delay is that a patient problem that is to be solved in order to achieve the target is not appropriately set. In general, in a rehabilitation hospital, a patient does rehabilitation with help from a therapist such as a physical therapist under the direction of a doctor. The therapist makes a rehabilitation plan, for example, in the below-shown manner: That is, the therapist implements rehabilitation under the guidance of a doctor in the below-shown manner. As being described below, in a flow of rehabilitation, after a target is set, rehabilitation may be implemented in order to achieve the target. That is, the therapist checks the target of rehabilitation (rehabilitation target) that is worked out at the time when the patient is admitted to the hospital, and then implements the rehabilitation in order to achieve the target.
(1) Set a target according to conditions and needs of the patient;
(2) Set a patient problem that should be solved in order to achieve the target;
(3) Determine rehabilitation to solve the problem; and
(4) Implement the rehabilitation.

For example, when a target that “To be able to climb up and down stairs” is set for a given patient, an element(s) that hinders the patient from climbing up and down stairs is set as a patient problem(s). In this example, when the patient is unable to climb up and down stairs due to the deterioration of a lower limb function, the “lower limb function” is set as a patient problem. Then, rehabilitation to improve the lower limb function is set. Further, the therapist deals with the patient problem in regard to the “lower limb function” by implementing this rehabilitation.

Note that since the conditions of the patient (hereinafter also referred to as patient conditions) changes day by day, the above-shown items (2) to (4) are repeatedly carried out on a daily basis according to the patient conditions. Further, since the rehabilitation is implemented in the above-described order, it becomes difficult to achieve the target if the “setting of the patient problem” is not appropriately made. This is because when the “setting of the patient problem” is not appropriately made, rehabilitation necessary for achieving the target may not be provided (e.g., indicated) to the patient. Therefore, it is important to appropriately make the setting of the patient problem.

Since the setting of the patient problem is usually made according to the empirical rule, it is difficult, especially for inexperienced therapists, to set the patient problem. Further, therapists are busy. Therefore, even when one therapist sets a patient problem in order to achieve the target of rehabilitation of a patient within a range indicated by a doctor, it is difficult to have another therapist check, on a daily basis, whether or not the patient problem is the best in achieving the target of the rehabilitation of the patient. Further, for example, in the case another therapist (such as a team leader) checks whether or not a patient problem set by an inexperienced therapist is the best in achieving the target of rehabilitation of the patient, it is conceivable that the other therapist checks the patient problem set in the electronic medical record of the patient. In that case, the other therapist needs to operate the electronic medical record, so that it is time-consuming. Therefore, it is desired to make it possible to automatically determine whether or not the patient problem is appropriately set and thereby to efficiently check whether or not the patient problem is appropriately set.

To deal with this, the support apparatus 1 according to the present disclosure is configured as described above, and therefore can output an alert when the matching level between the predicted patient problem and the actual patient problem is low, i.e., the difference between the predicted patient problem and the actual patient problem is large. Note that the actual patient problem is, for example, a patient problem that is dealt with according to the patient problem set by a therapist under the direction of a doctor. Further, the predicted patient problem is likely to be appropriate for the patient who does rehabilitation. Therefore, the situation where the difference between the predicted patient problem and the actual patient problem is large is a situation where there is a possibility that the set patient problem is not appropriate.

Therefore, the support apparatus 1 according to the present disclosure can automatically output an alert when there is a possibility that the set patient problem is not appropriate. In this way, the support apparatus 1 according to the present disclosure can efficiently check whether or not the patient problem is appropriately set. That is, the support apparatus 1 according to the present disclosure can support the evaluation of the set patient problem. Note that it is also possible to efficiently check whether or not the patient problem is appropriately set by using a support method performed by the support apparatus 1 or a program for performing such a support method.

As described above, according to this example embodiment, it is possible for a therapist to recognize that there is a possibility that the set patient problem is not appropriate without requiring time and effort. Then, in such a case, the therapist can deal with this matter so that an appropriate patient problem is set. For example, the therapist can recognize that the set patient problem may not be the best in achieving the target of rehabilitation of the patient even when it is within the range indicated by a doctor. Then, the therapist can deal with the matter so that an appropriate patient problem is set within the range indicated by the doctor. As a result, the target can be achieved as being planned and the discharge delay is prevented or reduced.

### First Example Embodiment

An example embodiment will be described hereinafter with reference to the drawings. For clarifying the explanation, the following description and the drawings are partially omitted and simplified as appropriate. Further, the same symbols are assigned to the same or corresponding components throughout the drawings, and redundant descriptions thereof are omitted as appropriate.

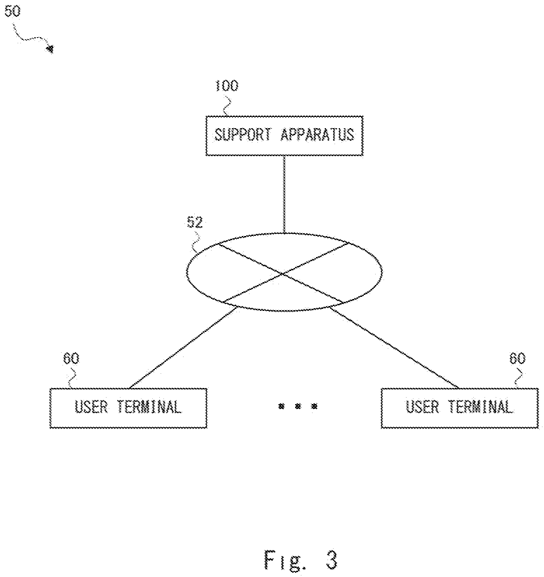

FIG. 3 shows a support system 50 according to a first example embodiment. The support system 50 includes at least one user terminal 60 and a support apparatus 100. The support apparatus 100 corresponds to the support apparatus 1 shown in FIG. 1. The user terminal 60 and the support apparatus 100 are connected to each other through a wired or wireless network 52 so that they can communicate with each other.

The support apparatus 100 is, for example, a computer such as a server or a personal computer. The support apparatus 100 supports the setting of a patient problem that should be solved in order to achieve a target in rehabilitation (capacity improving activity). Specifically, the support apparatus 100 performs control so that an alert is output when there is a possibility that the patient problem is not appropriately set.

The user terminal 60 is, for example, a computer. The user terminal 60 is, for example, a personal computer (PC) of a user such as a therapist, or a portable terminal, such as a tablet-type terminal or a smartphone, of a user. The user may enter patient information, i.e., information about a patient by using the user terminal 60. In such a case, the user terminal 60 receives the patient information through an input device. Then, the user terminal 60 transmits the patient information to the support apparatus 100. The support apparatus 100 stores the patient information. The patient information will be described later. Further, the user may also set a determined target, a patient problem, and rehabilitation by using user terminal 60. In this case, the user terminal 60 transmits information indicating the set target, the patient problem, and the rehabilitation to the support apparatus 100. Further, by using the user terminal 60, the user may enter, on a daily basis, patient conditions, a patient problem that has been dealt with (actual patient problem), and rehabilitation that has been implemented. In this case, the user terminal 60 transmits information indicating the patient conditions, information indicating the actual patient problem, and information indicating the implemented rehabilitation to the support apparatus 100.

Further, the user terminal 60 may receive an instruction to output an alert under the control of the support apparatus 100. In this case, the user terminal 60 makes an output apparatus (user interface), such as a display device, provided in the user terminal 60 output (display) an alert corresponding to the instruction received from the support apparatus 100.

Note that the support apparatus 100 may output an alert to a user interface provided in the support apparatus 100. Further, the alert may be displayed as an image on a display. Alternatively, the alert may be output by a lamp. Alternatively, the alert may be output by a sound or a voice. Alternatively, the alert may be output by vibrations of the user terminal 60 or the like.

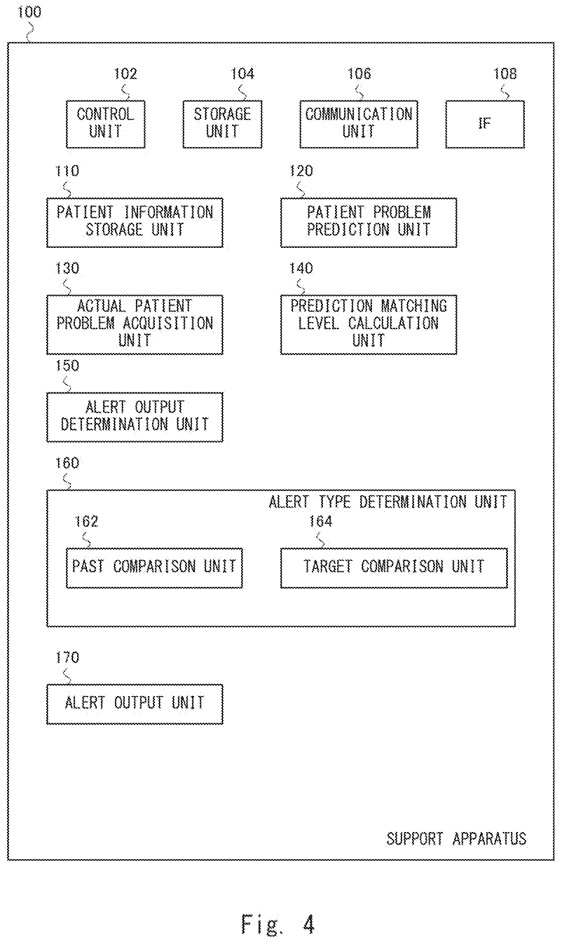

FIG. 4 shows a configuration of the support apparatus 100 according to the first example embodiment. The support apparatus 100 includes, as its main hardware configuration, a control unit 102, a storage unit 104, a communication unit 106, and interface (IF: Interface) unit 108. The control unit 102, the storage unit 104, the communication unit 106, and the interface unit 108 are connected to each other through a data bus or the like. Note that the user terminal 60 shown in FIG. 3 can also have the hardware configuration shown in FIG. 4.

The control unit 102 is a processor such as a CPU (Central Processing Unit). The control unit 102 has a function as an arithmetic apparatus that performs control processing, arithmetic processing, and the like. The storage unit 104 is, for example, a memory or a storage device such as a hard disk drive. The storage unit 104 is, for example, a ROM (Read Only Memory), a RAM (Random Access Memory) or the like. The storage unit 104 has a function of storing a control program(s), an arithmetic program(s), and the like performed by the control unit 102. Further, the storage unit 104 has a function of temporarily storing processing data and the like. The storage unit 104 can include a database.

The communication unit 106 performs processing necessary to communicate with the user terminal 60 (and other devices) through a network 52. The communication unit 106 can include communication ports, a router, a firewall, and the like. The interface (IF: Interface) unit 108 is, for example, a user interface (UI). The interface unit 108 includes an input device such as a keyboard, a touch panel, or a mouse, and an output device such as a display or a speaker(s). The interface unit 108 receives data input by a user (an operator) and outputs information for the user.

The support apparatus 100 according to the first example embodiment includes, as its main hardware configuration, a patient information storage unit 110, a patient problem prediction unit 120, an actual patient problem acquisition unit 130, a prediction matching level calculation unit 140, an alert output determination unit 150, an alert type determination unit 160, and an alert output unit 170. Further, the alert type determination unit 160 includes a past comparison unit 162 and a target comparison unit 164.

The patient information storage unit 110 has a function as patient information storage means. The patient problem prediction unit 120 corresponds to the prediction unit 2 shown in FIG. 1. The patient problem prediction unit 120 has a function as patient problem prediction means (prediction means). The actual patient problem acquisition unit 130 has a function as actual patient problem acquisition means (acquisition means). The prediction matching level calculation unit 140 corresponds to the calculation unit 4 shown in FIG. 1. The prediction matching level calculation unit 140 has a function as prediction matching level calculation means (calculation means, matching level calculation means).

The alert output determination unit 150 has a function as alert output determination means (output determination means). The alert type determination unit 160 has a function as alert type determination means (type determination means). The alert output unit 170 corresponds to the output unit 6 shown in FIG. 1. The alert output unit 170 has a function as alert output means (output means). Further, the past comparison unit 162 has a function as past comparison means. The target comparison unit 164 has a function as target comparison means.

Note that each of the above-described components can be implemented, for example, by executing a program under the control of the control unit 102. More specifically, each of the components can be implemented by having the control unit 102 execute a program stored in the storage unit 104. Further, each of the components can be implemented by recording a necessary program in an arbitrary non-volatile recording medium and installing it as required. Further, each of the components is not limited to those implemented by software using a program, and may be implemented by a combination of any two or more of hardware, firmware, and software. Further, each of the components may be implemented by using a user-programmable integrated circuit such as an FPGA (Field-Programmable Gate Array) or a microcomputer. In such a case, a program composed of a respective one of the above-described components may be implemented by using this integrated circuit. The above-described matters also apply to other example embodiments (which will be described later). Note that the specific function of each of the components will be described later.

By the above-described components, the support apparatus 100 predicts a patient problem that should be dealt with in order to achieve the target of the patient. Further, the support apparatus 100 calculates a matching level between a predicted patient problem and an actual patient problem. Further, the support apparatus 100 determines whether or not the calculated matching level is lower than a predetermined first threshold (alert output determination), and performs control so that an alert is output when the matching level is lower than the first threshold.

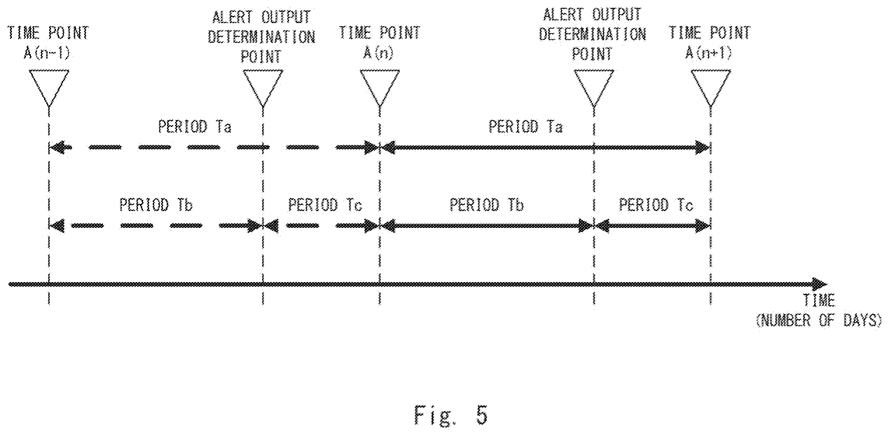

FIG. 5 is a diagram for explaining timings at which processes are performed by the support apparatus 100 according to the first example embodiment. FIG. 5 shows the lapse of time (elapsed days) in regard to rehabilitation. When the patient stays in the hospital, the lapse of time in regard to rehabilitation may correspond to the number of days of the hospitalization. In the example shown in FIG. 5, a patient problem is predicted for each period Ta from the start of the rehabilitation. These time points at each of which the patient problem is predicted are referred to as time points A. Further, an evaluation as to whether or not the target can be achieved as being planned may be made at each time point A (in each period Ta). Note that the period Ta is a predetermined period. The period Ta is, for example, but not limited to, 10 days. Further, an alert output determination is made after a period Tb has elapsed from the time point A. This time point after the period Tb has elapsed from the time point A is defined as an alert output determination time point. That is, it is determined whether or not to output an alert at the alert output determination time point. Then, when it is determined that the alert should be output, the alert is output at the alert output determination time point. Note that the period Tb is a predetermined period shorter than the period Ta. The period Tb is, for example, but not limited to, seven days. Further, a period Tc is a period from the alert output determination time point to the next time point A. Note that the relation Tc=Ta−Tb may hold.

The support apparatus 100 predicts, at the time point A (first time point), a patient problem that should be dealt with in a period between the time point A and a time point that is a period Ta (first time period) later than the time point A (i.e., in a period that starts at the time point A and has a length equal to the period Ta). That is, the support apparatus 100 calculates a predicted patient problem in the period Ta that starts at the time point A. Further, the support apparatus 100 acquires, at the alert output determination time point, a patient problem that has been actually dealt with (actual patient problem) in the period Tb (second period) that starts at the time point A. Then, the support apparatus 100 compares the predicted patient problem with the actual patient problem at the alert output determination time point, and thereby calculates a matching level (prediction matching level) between the actual patient problem and the predicted patient problem. Further, the support apparatus 100 determines whether or not the prediction matching level is lower than a predetermined threshold. When the prediction matching level is lower than the threshold, the support apparatus 100 performs control so that an alert indicating that the target may not be achieved because there is a possibility that the patient problem is not appropriately set is output.

By the above-described configuration, the support apparatus 100 can periodically make an alert output determination. Therefore, it is possible to efficiently check whether or not the patient problem is appropriately set at regular intervals. Further, since the alert output determination is made after the period Tb shorter than the period Ta has elapsed, it is possible to check whether or not the patient problem is appropriately set before the period Ta has elapsed from the time point A. Therefore, in the case where an evaluation as to whether or not the target can be achieved as being planned is made at each time point A, it is possible to check whether or not the patient problem is appropriately set before the evaluation is made.

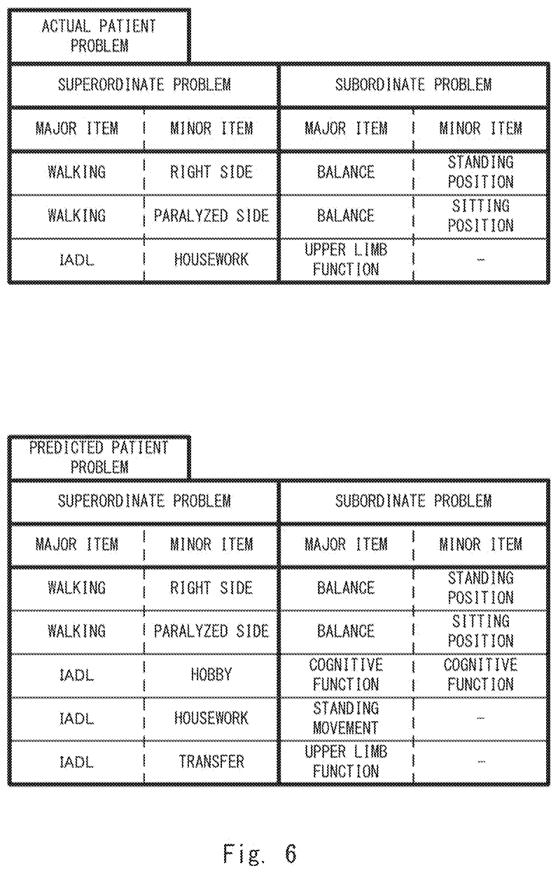

FIG. 6 shows an example of a predicted patient problem and an actual patient problem according to the first example embodiment. As shown in FIG. 6, each of the patient problems (predicted patient problem and actual patient problem) includes a plurality of items. Note that as shown as an example in FIG. 6, items included in a patient problem may be classified into a plurality of categories.

Further, items included in a patient problem may be classified into a superordinate problem and a subordinate problem. Note that the superordinate problem and the subordinate problem correspond to a superordinate concept and a subordinate concept, respectively, in regard to the problem. That is, the subordinate problem is a subordinate concept of the superordinate problem, and the superordinate problem is a superordinate concept of the subordinate problem. The superordinate problem indicates in what kind of movement the patient has a problem in his/her daily life. In other words, the superordinate problem indicates movements that the patient has difficulty in performing in his/her daily life. Further, the subordinate problem relates to causes of difficulty in performing movements in daily life indicated by the superordinate problem. The subordinate problem indicates impaired basic movements and functions of the body (such as a motor function and a cognitive function). That is, it is considered that since movements and functions corresponding to the subordinate problem are impaired, the patient has difficulty in performing movements indicted by the superordinate problem.

Further, items included in a patient problem may be classified into major items and minor items. That is, the items included in each of the superordinate problem and the subordinate problem may be further classified into major items and minor items. The major items indicate general problems, and the minor items indicate more specific problems. The major items indicate names of movements and names of physical functions, and the minor items indicate in what circumstances problems indicated in the major items occur in a specific manner.

The actual patient problem shown in FIG. 6 includes, as items in the superordinate problem, “Major Item: Walking, Minor Item: Right side”, “Major Item: Walking, Minor Item: Paralyzed side”, and “Major Item: IADL, Minor Item: Housework”. Further, the actual patient problem shown in FIG. 6 includes, as items in the superordinate problem, “Major Item: Balance, Minor Item: Standing position”, “Major Item: Balance, Minor Item: Sitting position”, and “Major item: Upper limb function, Minor item: None”. In this case, the patient of which the actual patient problem is shown above (i.e., the subject patient) has difficulty in “Walking (major item of superordinate problem) in regard to the “Right side (minor item of superordinate problem)” and the “Paralyzed side (minor item of superordinate problem)” in daily life. Further, as their causes, the function of “Balance (major item of subordinate problem)” in “Standing position (minor item of subordinate problem)” and “Sitting position (minor item of subordinate problem)” has been impaired. Further, the subject patient has difficulty in “IADL (major item of superordinate problem)” in regard to “Housework (minor item of superordinate problem)” in daily life. Further, as their causes, the “Upper limb function (major item of subordinate problem)” has been impaired. Note that as will be described later, the IADL stands for instrumental activities of daily living, i.e., comprehensive activities in daily living, and involves a complex movement and a movement requiring a decision.

Further, the predicted patient problem shown in FIG. 6 includes, as items in the superordinate problem, “Major Item: Walking, Minor Item: Right side”, “Major Item: Walking, Minor Item: Paralyzed side”, “Major Item: IADL, Minor Item: Hobby”, “Major Item: IADL, Minor Item: Housework”, and “Major Item: IADL, Minor Item: Transfer”. Further, the predicted patient problem shown in FIG. 6 includes, as items in the subordinate problem “Major Item: Balance, Minor Item: Standing position”, “Major Item: Balance, Minor Item: Sitting position”, “Major Item: Cognitive function; Major Item: Cognitive function”, “Major Item: Standing movement, Minor Item: None”, and “Major item: Upper limb function, Minor item: None”.

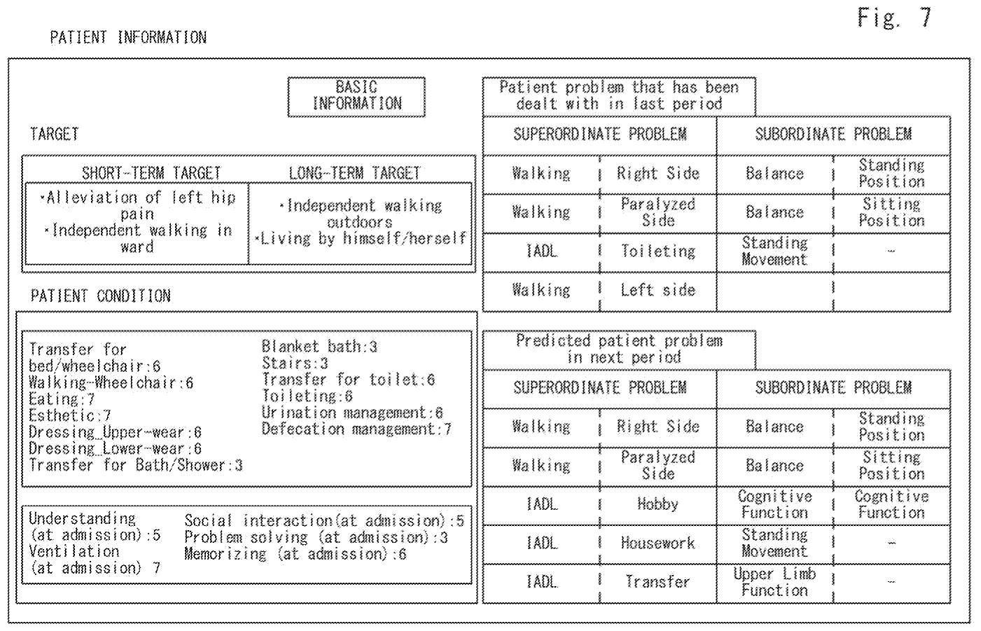

FIG. 7 shows an example of patient information according to the first example embodiment. The patient information may include basic information, patient conditions, and a set target of the subject patient. Further, the patient information may include a set patient problem and an actual patient problem. The basic information may indicate attributes of the subject patient. For example, the basic information may indicate a name, an age, and a gender of the subject patient. Further, the basic information may also include an expected period of hospitalization of the subject patient and the number of days he/she has stayed for in the hospital from the admission.

Further, the patient conditions may indicate functional levels of the patient, such as levels of motor functions and cognitive functions, at the present time. The functional levels may be, for example, ability values (ability levels) of the patient for daily-life activities. The functional levels may be, for example, scores (indicators) for ADL (Activities of Daily Living) or IADL (Instrumental Activities of Daily Living). Further, the functional levels may be indicated, for example, by evaluation scores of respective evaluation items in FIM (Function Independence Measure). Alternatively, the functional levels may be indicated by scores based on GCS (Glasgow Coma Scale) or JCS (Japan Coma Scale), or may be expressed by user's personal views.

In FIG. 7, the target includes short-term targets and long-term targets. In the example shown in FIG. 7, “Alleviation of left hip pain” and “Independent walking in ward” are set as short-term targets. Further, “Independent walking outdoors” and “Living by himself/herself” are set as long-term targets.

Note that the long-term target indicates the patient conditions and movements that the patient can perform, which are the target immediately before the discharge from the hospital in the rehabilitation plan. That is, the long-term target indicates the patient conditions that are desired to be achieved at the time of discharge from the hospital, and also indicates the patient conditions and movements that the patient can perform both of which are required to enable the patient to be discharged from the hospital to a place where the patient wishes to stay after the discharge (such as his/her house). Meanwhile, the short-term target indicates the patient conditions and movements that the patient can perform both of which are the target at a midpoint in the planned hospitalization period in the rehabilitation plan. That is, the short-term target indicates the patient conditions and movements that the patient can perform both of which should be achieved in the midpoint in order to achieve the long-term target.

Further, in the example shown in FIG. 7, the patient conditions are indicated by the current FIM. Specifically, FIM scores (degrees of independence) are associated with respective items indicating the patient conditions (e.g., “Transfer for bed/wheelchair”) as shown below. In this way, the patient conditions indicate how independent the patient currently is for various items.
7 points: Complete independence (including a time and safety)
6 points: Modified independence (independence with use of a help device)
5 points: Supervision (instruction, prompting, and preparation are required)
4 points: Minimal assistance (25% or lower assistance is required)
3 points: Moderate assistance (25% to 50% assistance is required)
2 points: Maximum assistance (50% to 75% assistance is required)
1 point: Total assistance (75% or higher assistance is required)

Further, as shown as an example in FIG. 7, the patient information may include a predicted patient problem predicted by the patient problem prediction unit 120 (which will be described hereinafter) and a patient problem that has been actually dealt with. In the example shown in FIG. 7, the patient information may include “Patient problem that has been dealt with in last period” and “Predicted patient problem in next period”. For example, when the present time is 20 days from the start of the rehabilitation, “Patient problem that has been dealt with in last period” may indicate a patient problem(s) that had been dealt with in days 10 to 19. Further, “Predicted patient problem in next period” may indicate a patient problem(s) that is predicted to be dealt with in days 20 to 29. Note that in FIG. 5, the present time corresponds to a “time point A (n)”, and the “last period” corresponds to a period Ta from a “time point A (n−1)” to a “time point A (n)”. Further, the “next period” corresponds to a period Ta from the “time point A (n)” to a “time point A (n+1)”.

In the example shown in FIG. 7, “Predicted patient problem in next period” is the same as that shown in FIG. 6. Further, in the example shown in FIG. 7, “Patient problem that has been dealt with in last period” includes, as items in the superordinate problem, “Major Item: Walking, Minor Item: Right side”, “Major Item: Walking, Minor Item: Paralyzed side”, “Major Item: IADL, Minor Item: Toileting”, and “Major Item: Walking, Minor Item: Left side”. Further, “Patient problem that has been dealt with in last period” includes, as items in the subordinate problem, “Major Item: Balance, Minor Item: Standing position”, “Major Item: Balance, Minor Item: Sitting position”, and “Major Item: Standing movement, Minor Item: None”. In this case, the subject patient has difficulty in “Walking (major Item of superordinate problem)” in regard to “Right side (minor item of superordinate problem)”, “Paralyzed side (minor item of superordinate problem)”, and “Left side (minor item of superordinate problem)” in daily life. Further, as their causes, the function of “Balance (Major Item of subordinate problem)” in “Standing position (minor item of subordinate problem)” and “Sitting position (minor item of subordinate problem)” has been impaired. Further, the subject patient has difficulty in “IADL (major item of superordinate problem)” in regard to “Toileting (minor item of superordinate problem)” in daily life. Further, as their causes, “Standing movement (major item of subordinate problem)” has been impaired.

Note that the support apparatus 100 may perform control so that the patient information shown in FIG. 7 is displayed. In this case, the support apparatus 100 may perform control so that the patient information shown in FIG. 7 is displayed for each period Ta. Further, in this case, the support apparatus 100 may perform control so that the patient information is displayed independently of an alert. Further, the support apparatus 100 may perform control so that the patient information is displayed on a display device (user interface) of the user terminal 60. Further, the support apparatus 100 may perform control so that the patient information is displayed in the interface unit 108.

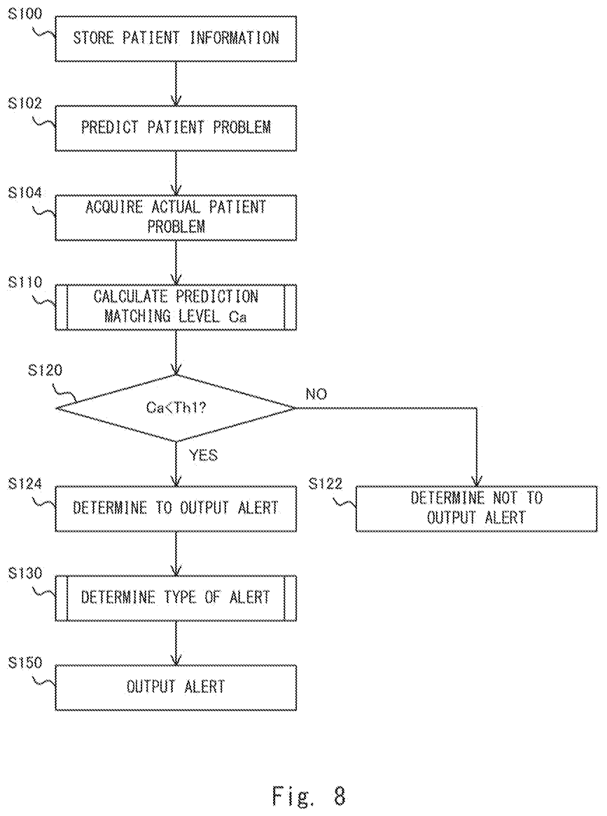

FIG. 8 is a flowchart showing a support method performed by the support apparatus 100 according to the first example embodiment. The processes shown in the flowchart shown in FIG. 8 may be performed for each patient (subject patient) which is a subject to be processed. The patient information storage unit 110 stores patient information (Step S100). Specifically, the patient information storage unit 110 stores patient information in the storage unit 104. The patient information storage unit 110 may store, for example, patient information received from the user terminal 60. Further, the patient information storage unit 110 may store the patient information shown in FIG. 7.

The patient problem prediction unit 120 predicts a patient problem (Step S102). Specifically, the patient problem prediction unit 120 calculates, as a predicted problem of the subject patient (predicted patient problem), a patient problem that had been actually dealt with in the past for a patient (past patient) related to patient information that is similar to the patient information of the subject patient. Note that the patient problem prediction unit 120 may determine that the patient information of the subject patient and that of the past patient are similar to each other when the degree of similarity between them is equal to or greater than a predetermined threshold. The degree of similarity may be calculated by, for example, comparing basic information of the subject patient with that of the past patient, comparing the target of the subject patient with that of the past patient, and comparing the patient conditions of the subject patient with those of the past patient. Further, the degree of similarity may be calculated by comparing the degree of recovery of the subject patient with that of the past patient.

Further, as described above with reference to FIG. 5, the patient problem prediction unit 120 calculates a predicted patient problem in a period Ta (first period) that starts at a time point A (first time point). Specifically, the patient problem prediction unit 120 calculates, as the predicted patient problem, a patient problem that had been actually dealt with in a period Ta that starts at a time point when the patient information of the past patient, who is related to the patient information similar to the patient information of the subject patient obtained at the time point A, was obtained for the past patient.

Note that the patient problem prediction unit 120 does not necessarily have to predict the patient problem at the time point A. The patient problem prediction unit 120 may predict the patient problem that should be dealt with in the period Ta at the alert output determination time point. Even in this case, the patient problem prediction unit 120 predicts the patient problem by using the patient information obtained at the time A earlier than the alert output determination time point.

The actual patient problem acquisition unit 130 acquires an actual patient problem of the subject patient (Step S104). Specifically, the actual patient problem acquisition unit 130 acquires an actual patient problem entered by the therapist. The actual patient problem acquisition unit 130 may acquire (receive) an actual patient problem from the user terminal 60 of the therapist. Alternatively, the actual patient problem acquisition unit 130 may acquire (extract) an actual patient problem stored in the support apparatus 100. In this case, the actual patient problem may be contained in the patient information stored in the patient information storage unit 110. Further, as described above with reference to FIG. 5, at the alert output determination time point, the actual patient problem acquisition unit 130 acquires a patient problem that had been actually dealt with in a period Tb (second period) that starts at a time point A (i.e., an actual patient problem). Note that the therapist actually deals with a predefined patient problem in daily work and enters the patient problem that he/she actually dealt with into the user terminal 60 or the like. Therefore, the actual patient problem can correspond to the set patient problem.

The prediction matching level calculation unit 140 calculates a prediction matching level Ca, which is a matching level between the actual patient problem and the predicted patient problem (Step S110). Specifically, the prediction matching level calculation unit 140 compares the actual patient problem and the predicted patient problem at the alert output determination time point shown in FIG. 5, and thereby calculates the prediction matching level Ca. Note that the prediction matching level Ca indicates the degree of matching between the actual patient problem and the predicted patient problem. That is, the prediction matching level Ca indicates how much the actual patient problem and the predicted patient problem match each other. That is, the prediction matching level Ca becomes small when a difference between the actual patient problem and the predicted patient problem is large, and becomes large when the difference between the actual patient problem and the predicted patient problem is small. An example of a method for calculating a prediction matching level Ca will be described hereinafter, but the method for calculating a prediction matching level Ca is not limited to the below-shown example.

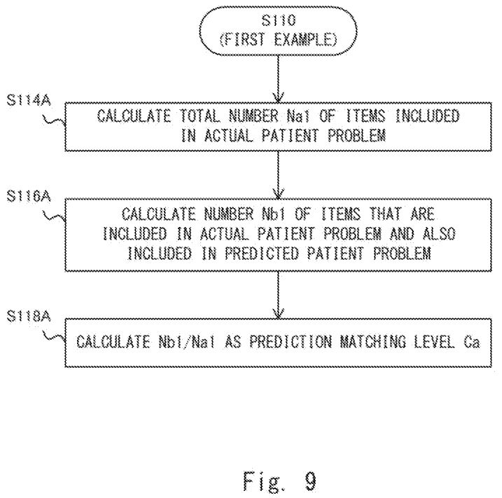

FIG. 9 is a flowchart showing a first example of the method for calculating a prediction matching level Ca (S110) performed by the prediction matching level calculation unit 140 according to the first example embodiment. The prediction matching level calculation unit 140 calculates the total number Na1 of items included in the actual patient problem (Step S114A). In the example shown in FIG. 6, for example, the prediction matching level calculation unit 140 calculates the total number of items included in the actual patient problem as six (items) (Na1=6). Note that in this example, the items in the patient problem that are compared when a prediction matching level is calculated may be those that are included in either of the major and minor items. That is, the items in the patient problem that are compared when a prediction matching level is calculated may correspond to those that are included in the combination of the major and minor items.

Next, the prediction matching level calculation unit 140 calculates the number Nb1 of items that are included in the actual patient problem and also included in the predicted patient problem (Step S116A). In the example shown in FIG. 6, since all the items included in the actual patient problem are also included in the predicted patient problem, Nb1 is calculated as six (items) (Nb1=6). Next, the prediction matching level calculation unit 140 calculates Nb1/Na1 as the prediction matching level Ca (Step S118A). That is, in the first example, the prediction matching level Ca is defined as Nb1/Na1 (Ca=Nb1/Na1). In other words, the prediction matching level calculation unit 140 calculates, as the prediction matching level Ca, the ratio of the number Nb1 of items that are included in the actual patient problem and also included in the predicted patient problem to the total number Na1 of items included in the actual patient problem. In the example shown in FIG. 6, for example, the prediction matching level calculation unit 140 calculates that the prediction matching level Ca is equal to 1.0 (Ca=6/6=1.0). In the first example, the prediction matching level Ca can be calculated by a method simpler than the method in the second example (which will be described later).

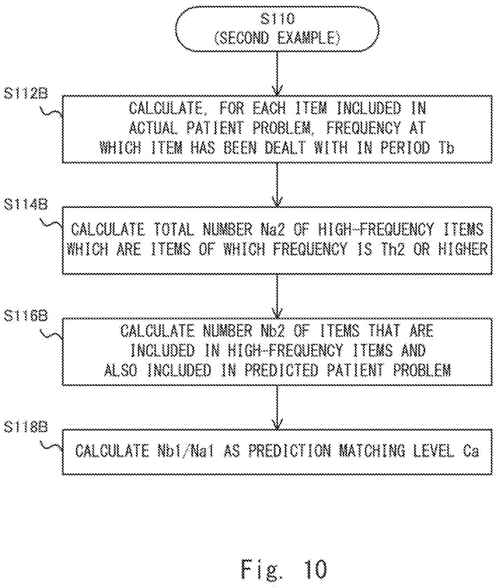

FIG. 10 is a flowchart showing a second example of the method for calculating a prediction matching level Ca (S110) performed by the prediction matching level calculation unit 140 according to the first example embodiment. The prediction matching level calculation unit 140 calculates, for each item included in the actual patient problem, a frequency at which the item has been dealt with in a period Tb (Step S112B). For each item, the larger number of times the item is dealt with in the period Tb, the higher the frequency at which the item is dealt with becomes.

There are various methods for calculating a frequency at which an arbitrary item A included in the actual patient problem is dealt with. For example, the frequency may be expressed as Frequency=(Number of days in period Tb on each of which item A was dealt with)/(Number of days in period Tb). That is, the frequency of the item A may be the ratio of the number of days in the period Tb on each of which the item A was dealt with to the total number of days in the period Tb. In this case, for example, assuming that the number of days in the period Tb is seven and the number of days in the period Tb on each of which the item A was dealt with is four, the frequency of the item A is calculated as 0.57 (4/7≈0.57).

Alternatively, the frequency of the item A may be calculated as Frequency=(Number of times item A was dealt with in period Tb)/(Total number of times in period Tb). That is, the frequency of the item A may be the ratio of the number of times the item A was dealt with to the total number of times in the period Tb. Note that the “Total number of times in the period Tb” is the sum total of the number of times any of the items was dealt with in the period Tb. For example, assuming that the number of days in the period Tb is seven, and some patient problem is dealt with nine times in total every day for seven days, the “Total number of times in the period Tb” is 63 times (9×7=63). Further, it is assumed that the item A has been dealt with 21 times in the period Tb. In this case, the frequency of the item A is calculated as 0.33 (21/63≈0.33).

Next, the prediction matching level calculation unit 140 calculates the total number Na2 of high-frequency items (Step S114B). Note that the high-frequency item is an item of which the frequency is equal to or higher than a predetermined threshold Th2. For example, when the total number of items in the actual patient problem is 20 and the frequencies of five of these items are equal to or higher than the threshold Th2, the total number Na2 of high-frequency items (first items) is calculated as five (Na2=5). Note that the threshold Th2 (second threshold) is, for example, but not limited to, 0.5 (Th2=0.5). Further, in the above-described example of the method for calculating a frequency, when the frequency is expressed as Frequency=(Number of days in period Tb on each of which item A was dealt with)/(Number of days in period Tb), the threshold Th2 may be 0.5 (Th2=0.5). In contrast, when the frequency is expressed as Frequency=(Number of times item A was dealt with in period Tb)/(Total number of times in period Tb), the threshold Th2 may be 0.3 (Th2=0.3).

Note that in the above-described example, the frequency of the item A is the ratio of the number of days on each of which the item A is dealt with (or the number of times the item A is dealt with) to the total number of days in the period Tb (or the number of times in the period Tb), but the frequency is not limited to this example. When the number of days in the period Tb is determined in advance, the frequency may simply be the number of days in the period Tb on each of which the item A is dealt with. In this case, for example, the threshold Th2 may be four (days) (Th2=4). Alternatively, when the total number of times in the period Tb is determined in advance, the frequency may simply be the number of times the item A is dealt with in the period Tb. In this case, for example, the threshold Th2 may be 20 (times) (Th2=20).

Next, the prediction matching level calculation unit 140 calculates the number Nb2 of items that are included the high-frequency items and also included in the predicted patient problem (Step S116B). For example, when there are two items that are included in the five high-frequency items and also included in the predicted patient problem, the number Nb2 is calculated as two (items) (Nb2=2). Next, the prediction matching level calculation unit 140 calculates Nb2/Na2 as the prediction matching level Ca (Step S118B). That is, in the second example, the prediction matching level Ca is defined as Nb2/Na2 (Ca=Nb2/Na2). That is, the prediction matching level calculation unit 140 calculates, as the prediction matching level Ca, the ratio of the number Nb2 of high-frequency items, i.e., items of each of which the frequency at which the item is dealt with in the period Tb is equal to or higher than the threshold Th2, that are included in the actual patient problem and also included in the predicted patient problem to the total number Na2 of high-frequency items. In the above-described example, the prediction matching level calculation unit 140 calculates the prediction matching level Ca as 0.4 (Ca=2/5=0.4).

The flowchart shown in FIG. 8 will be explained again. The alert output determination unit 150 determines whether or not the prediction matching level Ca calculated in the process in the step S110 is lower than a threshold Th1 (Step S120). Note that the threshold Th1 (first threshold) has a predetermined value. For example, the threshold Th1 is 0.5 (Th1=0.5), but the threshold is not limited to this example.

When the prediction matching level Ca is not lower than the threshold Th1 (No in S120), the alert output determination unit 150 determines to output no alert (Step S122). In this case, the flow of processes may be finished. Note that this alert corresponds to an alert indicating that the target may not be achieved because there is a possibility that the patient problem is not appropriately set. On the other hand, when the prediction matching level Ca is lower than the threshold Th1 (Yes in S120), the alert output determination unit 150 determines to output an alert (Step S124).

Note that the predicted patient problem is a patient problem that was dealt with in the past for a past patient whose conditions are similar to those of the subject patient. Therefore, it is expected that the target of the subject patient is likely to be achieved by coping with the items included in the predicted patient problem. Therefore, the predicted patient problem can be a model patient problem. In contrast, when the matching level between the actual patient problem and the predicted patient problem (prediction matching level) is small, the actual patient problem has deviated from the predicted patient problem. Therefore, the actual patient problem is likely to be one that is far from the predicted patient problem which can serve as the model. Then, as described above, since the therapist is actually coping with the predefined patient problem, the actual patient problem corresponds to the set patient problem. Therefore, when the prediction matching level is small, there is a possibility that the patient problem is not appropriately set, so that the target may not be achieved unless the patient problem is changed. Therefore, the support apparatus 100 according to the first example embodiment outputs, when the prediction matching level is small, an alert indicating that the target may not be achieved because there is a possibility that the patient problem is not appropriately set.

When it is determined that an alert should be output (Step S124), the alert type determination unit 160 determines the type of the alert (Step S130). Note that the “type of the alert” indicates why the alert is output. In other words, the type of the alert may correspond to the cause of the deviation between the actual patient problem and the predicted patient problem. Further, the type of the alert indicates under what circumstances it is determined that the alert should be output. In other words, the type of the alert may correspond to the type of the situation because of which the deviation has occurred between the actual patient problem and the predicted patient problem.

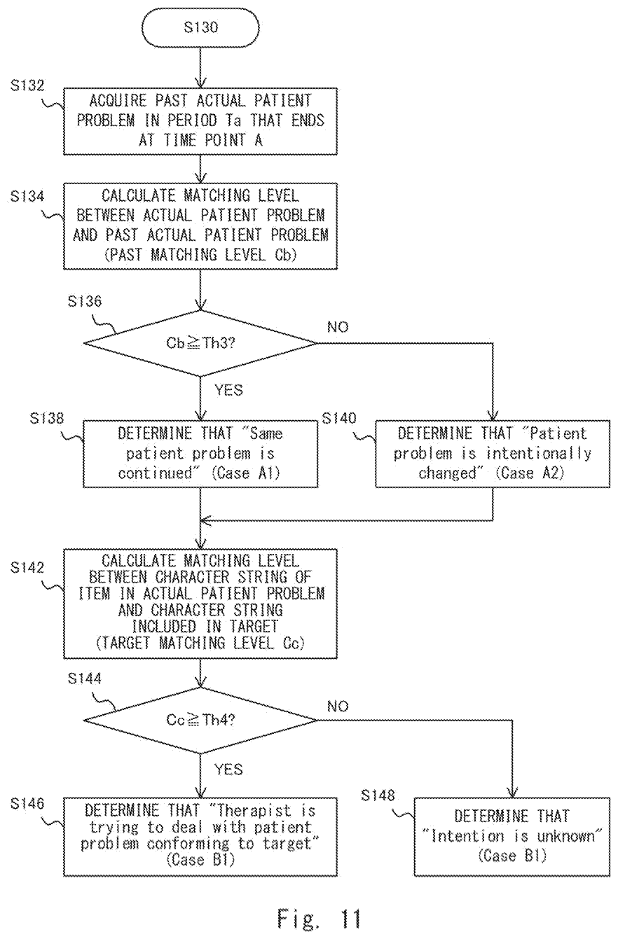

FIG. 11 is a flowchart showing a method for determining a type of an alert performed by the alert type determination unit 160 according to the first example embodiment (S130). The past comparison unit 162 of the alert type determination unit 160 determines the type of the alert according to a comparison between an actual patient problem and a patient problem that had been actually dealt with in a period earlier than the period in which the actual patient problem was dealt with (i.e., a past actual patient problem) (S132 to S140). Further, the target comparison unit 164 of the alert type determination unit 160 determines the type of the alert according to a comparison between a character string of an item included in the actual patient problem and a character string of a word included in the target (S142 to S148).

The past comparison unit 162 acquires a past actual patient problem in a period Ta that starts a time point earlier than the time point A and ends at the time point A (Step S132). That is, in FIG. 5, the past comparison unit 162 acquires a patient problem that had been actually dealt with in a period between a time point A (N−1), which is a period Ta earlier than the time point A (N) (i.e., which is earlier than the time point A (N) by a period Ta), and the time point A (N) (i.e., a past actual patient problem). The method for acquiring a past actual patient problem may be substantially the same as the method for acquiring an actual patient problem in the step S104.

The past comparison unit 162 calculates a matching level (past matching level Cb) between the actual patient problem and the past actual patient problem (Step S134). The method for calculating a past matching level Cb may be, for example, substantially the same as the above-described first example of the method for calculating a prediction matching level. That is, the past matching level Cb may be a matching level between the actual patient problem and the past actual patient problem. Specifically, the past comparison unit 162 calculates the total number Na3 (=Na1) of items included in the actual patient problem. The past comparison unit 162 calculates the number Nb3 of items that are included in the actual patient problem and also included in the past actual patient problem. The past comparison unit 162 calculates Nb3/Na3 as the past matching level Cb. That is, the past comparison unit 162 calculates, as the past matching level Cb, the ratio of the number of items that are included in the actual patient problem and also included in the past actual patient problem to the total number of items included in the actual patient problem.

The past comparison unit 162 determines whether or not the past matching level Cb is equal to or higher than a threshold Th3 (Step S136). Note that the threshold Th3 (third threshold) has a predetermined value. The threshold Th3 is, for example, but not limited to, 0.5 (Th3=0.5). When the past matching level Cb is equal to or higher than the threshold Th3 (Yes in S136), the deviation between the actual patient problem and the past actual patient problem is small. Therefore, in this case, the past comparison unit 162 determines that the therapist “is continuing the same patient problem” for the subject patient (Step S138). This case in the step S138 is referred to as “Case A1”. On the other hand, when the past matching level Cb is lower than the threshold Th3 (No in S136), the deviation between the actual patient problem and the past actual patient problem is large. Therefore, in this case, the past comparison unit 162 determines that the therapist “has intentionally changed the patient problem” for the subject patient (Step S140). This case in the step S140 is referred to as “Case A2”.

As described above, the past comparison unit 162 is configured to determine the type of an alert according to the comparison between the actual patient problem and the past actual patient problem. In this way, the therapist or the like can easily recognize whether the alert is output because he/she is continuing the same patient problem or the alert is output because he/she has intentionally changed the patient problem.

The target comparison unit 164 calculates a matching level (target matching level Cc) between a character string of an item in the actual patient problem and a character string included in the target (Step S142). The method for calculating a target matching level Cc may be, for example, substantially the same as the above-described first example of the method for calculating a prediction matching level. That is, the target matching level Cc may be the degree of matching (the degree of similarity) between a character string of an item included in the actual patient problem and a character string of a word included in the target.

Further, the target comparison unit 164 may determine whether or not a character string of an item included in the actual patient problem is also included in a word in the target. For example, in the example shown in FIGS. 6 and 7, a character string of an item “Walking” in the actual patient problem is also included in a target (short-term target and long-term target) shown in FIG. 7. Therefore, the target comparison unit 164 determines that the item “Walking” in the actual patient problem matches the word in the target.

Further, the target comparison unit 164 may determine whether or not a character string similar to a character string of an item included in the actual patient problem is also included in a word in the target. For example, in the example shown in FIGS. 6 and 7, the character string of the item “IADL” in the actual patient problem is similar to the word “life” included in the target (short-term target and long-term target) shown in FIG. 7. Therefore, the target comparison unit 164 determines that the item “IADL” in the actual patient problem is similar to the word in the target. Note that the determination as to whether a word is similar or not can be made by an existing word similarity determination method. For example, the target comparison unit 164 performs a morphological analysis on the target and decomposes the target into words. Then, the target comparison unit 164 may convert the words in the target and in the actual patient problem into vectors, determine the degree of similarity (degree of cosine similarity) of the obtained word vectors and thereby determine whether the words are similar or not to each other. Alternatively, the target comparison unit 164 may determine whether the word is similar or not by using a similar word dictionary created in advance.

The target comparison unit 164 calculates the total number Na4 (=Na1) of items included in the actual patient problem. Further, the target comparison unit 164 calculates the number Nb4 of items that are included in the actual patient problem and of each of which the character string matches (or is similar to) a character string of a word included in the target. The target comparison unit 164 calculates Nb4/Na4 as the target matching level Cc. That is, the target comparison unit 164 calculates, as the target matching level Cc, the ratio of the number of items that are included in the actual patient problem and of each of which the character string is the same as a character string of a word included in the target to the total number of items included in the actual patient problem.

The target comparison unit 164 determines whether or not the target matching level Cc is equal to or higher than a threshold Th4 (Step S144). Note that the threshold Th4 (fourth threshold) has a predetermined value. The threshold Th4 is, for example, but not limited to, 0.3 (Th4=0.3). When the target matching level Cc is equal to or higher than the threshold Th4 (Yes in S144), a lot of character strings of items included in the actual patient problem or character strings similar thereto are also included in the target. Therefore, in this case, the target comparison unit 164 determines that the therapist is “trying to deal with the patient problem that conforms to the target” for the subject patient (Step S146). This case in the step S146 is referred to as “Case B1”. On the other hand, when the target matching level Cc is lower than the threshold Th4 (No in S144), no or a few character strings of items included in the actual patient problem or character strings similar thereto are included in the target. Therefore, the therapist's intention of coping with the patient problem is unknown. Therefore, the target comparison unit 164 determines that the “intention is unknown” (Step S148). This case in the step S148 is referred to as “Case B2”.

As described above, the target comparison unit 164 is configured to determine the type of an alert according to the comparison between the character string of an item included in the actual patient problem and the character string of a word included in the target. In this way, the therapist or the like can easily recognize whether or not the alert is output even though he/she is trying to deal with the patient problem conforming to the target.

The flowchart shown in FIG. 8 will be explained again. The alert output unit 170 performs control so as to output an alert (Step S150). Note that the alert output unit 170 may perform control so that the interface unit 108 displays an alert. Alternatively, the alert output unit 170 may perform control so that the user terminal 60 displays an alert. In this case, the alert output unit 170 may transmit an instruction to display an alert (alert output instruction) to the user terminal 60. As a result, the user terminal 60 makes an output device (user interface) such as a display device provided in the user terminal 60 display an alert corresponding to the alert output instruction.

Alternatively, the alert output unit 170 may perform control so as to output an alert by a sound, a voice, or the like. Even in this case, the alert output unit 170 may perform control so that the interface unit 108 or the user terminal 60 outputs an alert by a sound, a voice, or the like. In this case, the alert output unit 170 may transmit an instruction (alert output instruction) to output an alert by a sound or a voice to the user terminal 60. As a result, the user terminal 60 outputs an alert corresponding to the alert output instruction by activating a speaker provided in the user terminal 60.

Alternatively, the alert output unit 170 may perform control so as to output an alert by vibrations or the like. In this case, the alert output unit 170 may transmit an instruction (alert output instruction) to output an alert by vibrations to the user terminal 60. As a result, the user terminal 60 outputs an alert corresponding to the alert output instruction by activating a vibration function provided in the user terminal 60.

Further, the alert output unit 170 may perform control so as to output an alert for each patient. Further, the alert output unit 170 may perform control so as to output an alert in a different form according to the type of the alert determined in the process in the step S130. For example, the alert output unit 170 may perform control so as to output an alert the display of which is different according to the type of the alert.

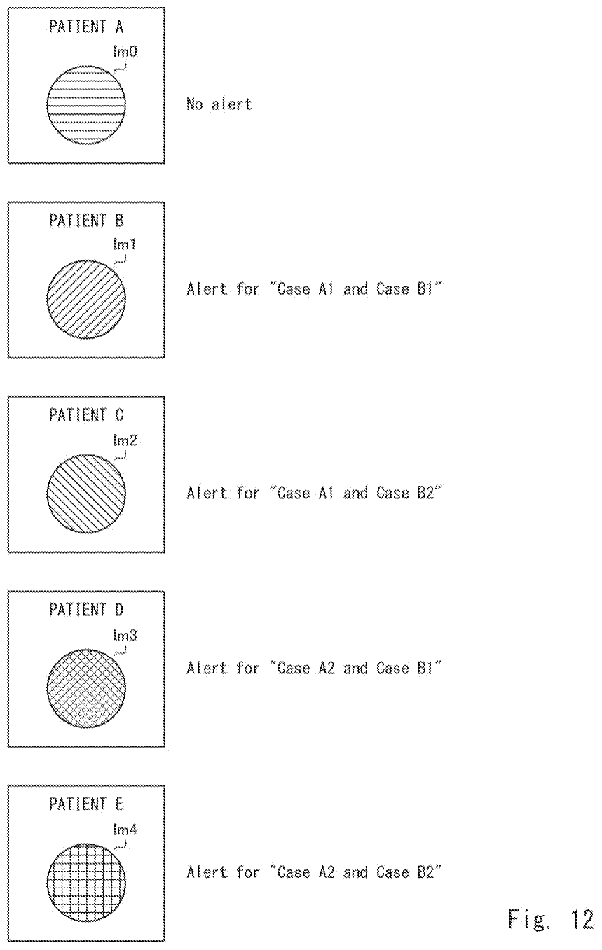

FIG. 12 shows examples of alerts output under the control of the alert output unit 170 according to the first example embodiment. FIG. 12 shows an example in which alerts are displayed in colors. That is, in the example shown in FIG. 12, an alert is displayed in a color on a display or by a lamp. Further, in the example shown in FIG. 12, an alert is displayed by a different color according to the type of the alert. Note that as shown in FIG. 12, an alert may be output for each patient. That is, an alert may be output in association with a respective patient. In this case, an alert may be output together with the patient information shown in FIG. 7. For example, when patient information drawn like the one shown in FIG. 7 is displayed on a screen, an image indicating an alert may be displayed in a margin part (e.g., an upper-left part) of the screen on which the patient information is displayed.

In the example shown in FIG. 12, an alert image Im0 indicating “No alert” is displayed for a patient A. The alert image Im0 is shown, for example, in blue. In this case, the patient problem set for the patient A may have been appropriately set.

Further, an alert image Im1 showing an alert of “Case A1 and Case B1” is displayed for a patient B. The alert image Im1 is shown, for example, in red. In this case, the patient problem set for the patient B is in a situation of “Case A1 and Case B1”, and hence may not have been appropriately set.

Further, an alert image Im2 showing an alert of “Case A1 and Case B2” is displayed for a patient C. The alert image Im2 is shown, for example, in orange. In this case, the patient problem set for the patient C is in a situation of “Case A1 and Case B2”, and hence may not have been appropriately set.

Further, an alert image Im3 showing an alert of “Case A2 and Case B1” is displayed for a patient D. The alert image Im3 is shown, for example, in dark red. In this case, the patient problem set for the patient D is in a situation of “Case A2 and Case B1”, and hence may not have been appropriately set.

Further, an alert image Im4 showing an alert of “Case A2 and Case B2” is displayed for a patient E. The alert image Im4 is shown, for example, in purple. In this case, the patient problem set for the patient E is in a situation of “Case A2 and Case B2”, and hence may not have been appropriately set.

Note that although it is assumed that an alert is output in a different color according to the type of the alert in the example shown in FIG. 12, the configuration according to the present disclosure is not limited to such a configuration. An alert may be displayed as a message such as a character string. In this case, for the patient B in the example shown in FIG. 12, a message such as “An alert indicating Case A1 and Case B1 has been issued for the patient B” may be displayed. Further, when the alert is output by a voice, the above-described message may be output by a voice. Further, when the alert is output by an alarm sound, a different alarm sound may be output according to the type of the alert. Further, when the alert is output by vibrations, vibrations in a different vibration pattern may be generated according to the type of the alert. Alternatively, an alert indicating “Case A1 and Case B1” may be output by a sound or a voice, and an alert indicating “Case A1 and Case B2” may be output by vibrations.

As described above, the support apparatus 100 according to the first example embodiment is configured so as to output an alert when the matching level between the actual patient problem and the predicted patient problem is small. In this way, when there is a difference between the predicted patient problem, which can serve as a model patient problem, and the actual patient problem, and hence there is a possibility that the patient problem is not appropriately set, an alert is output. Therefore, it is possible to efficiently check whether or not the patient problem is appropriately set.

Note that it is preferred that a leader (such as a team leader) of the therapist in charge of the patient for whom the alert has been output, instead of the therapist in charge of the patient, checks the alert. That is, the alert is preferably output to the user terminal 60 of the team leader. In this way, the team leader can provide appropriate advice and guidance to the therapist in charge. Further, since an alert is output in a different form according to the type of the alert, the team leader can recognize the type of the alert more easily, and hence can provide more appropriate advice.

For example, when an alert related to Case A1 is output, the team leader can point out, to the therapist in charge of the patient for whom the alert has been output, that “You are repeating the same patient problem, but don't you have to deal with other patient problems?”. Further, for example, when an alert related to Case A2 is output, the team leader can point out, to the therapist in charge of the patient for whom the alert has been output, “You seem to be dealing with a patient problem different from the one you dealt with the last time. Why?”. Further, when an alert related to Case B1 is output, the team leader can recognize that although the therapist is trying to deal with a patient problem conforming to the target, the patient problem may not be appropriate.

Note that when an alert is output to the user terminal 60 of the therapist in charge (especially an inexperienced therapist in charge), there is a risk that the therapist in charge may believe the alert blindly and modify the patient problem without careful consideration. Further, a therapist often sets a patient problem while taking, through a conversation with the patient and the observation of the patient's conditions, circumstances of the patient (information about the patient and external factors and the like) that do not appear in data into consideration. Therefore, it is considered that, for example, the therapist should respect the patient problem, which he/she considers to be necessary to achieve the target while facing the patient and under the direction of a doctor, as much as possible. Therefore, it is expected that by configuring the system or the like so that an alert is output to the user terminal 60 of the team leader, the team leader discusses with the therapist in charge, so that a more appropriate patient problem is set.

Note that although this example embodiment is configured so as to predict a patient problem based on data in the past, the result of this prediction is preferably not presented to the therapist in charge for the following reasons. A patient problem which the therapist has examined while taking the patient's circumstances into consideration under the direction of a doctor should be respected as much as possible as described above, so it is not desirable that the therapist in charge believes the prediction result blindly and changes the patient problem without careful consideration. Further, when the prediction result is presented to the therapist in charge, the therapist in charge may set the patient problem according to the prediction result, so there is a risk that the therapist in charge does not carefully consider the patient problem. Therefore, it is preferred not to present the prediction result to the therapist, especially to those who do not have much experience, also from the point of view of education.

Further, as described above, in the second example of the method for calculating a prediction matching level, the prediction matching level is calculated for high-frequency items that are included in the actual patient problem and have been frequently dealt with in the period Tb. For example, assume that the actual patient problem includes five items including three high-frequency items. Further, assume that the predicted patient problem includes two high-frequency items of the five items included in the actual patient problem. Further, assume that the threshold Th1 is 0.5 (Th1=0.5). In this case, in the first example, the prediction matching level becomes 0.4 (2/5=0.4), which is lower than the threshold Th1, so that an alert is output. In contrast, in the second example, the prediction matching level becomes 0.67 (2/3≈0.67), which is higher than the threshold Th1, so that no alert is output. As descried above, even when the actual patient problem and the predicted patient problem in the second example are the same as those in the first example, the results of them can be different from each other.

Note that since the high-frequency items are items that the therapist in charge has tried to deal with a lot of times, these items are items that reflect the intention of the therapist in charge. Therefore, by calculating a matching level between the actual patient problem and the predicted patient problem for the high-frequency items, it is possible to determine whether the intention of the therapist in charge is appropriate to achieve the target or not more effectively. That is, when the prediction matching level calculated for the high-frequency items is low, it is likely that the therapist in charge is repeating an ineffective patient problem a lot of times, and hence it is likely that the intention of the therapist in charge is not appropriate to achieve the target. On the other hand, when the prediction matching level calculated for the high-frequency items is high, it is likely that the therapist in charge is repeating an effective patient problem a lot of times, and hence it is likely that the intention of the therapist in charge is appropriate to achieve the target. Therefore, by calculating a prediction matching level as being calculated in the second example, it is possible to determine whether or not to output an alert while reflecting the intention of the therapist therein. That is, it is possible to output an alert when there is a possibility that the intention of the therapist is not appropriate to achieve the target.

### Second Example Embodiment

Next, a second example embodiment will be described with reference to the drawings. For clarifying the explanation, the following description and the drawings have been partially omitted and simplified as appropriate. Further, the same symbols are assigned to the same or corresponding components throughout the drawings and redundant descriptions thereof are omitted as appropriate. Note that since a system configuration according to the second example embodiment is substantially the same as that shown in FIG. 3, the description thereof is omitted. Further, since the configuration of a support apparatus 100 according to the second example embodiment is substantially the same as that shown in FIG. 4, the description thereof is omitted.

As shown in FIG. 6, items included in a patient problem can be classified into a plurality of categories. The support apparatus 100 according to the second example embodiment calculates a matching level for each category of the patient problem and performs control so as to output an alert for each category of the patient problem. That is, the prediction matching level calculation unit 140 according to the second example embodiment calculates a matching level for each category of the patient problem. Further, the alert output unit 170 according to the second example embodiment performs control so as to output an alert for each category of the patient problem. Note that the “major item of superordinate problem” is referred to as “Category A”; the “minor item of superordinate problem” is referred to as “Category B”; the “major item of subordinate problem” is referred to as “Category C”; and the “minor item of subordinate problems” is referred to as “Category D”.

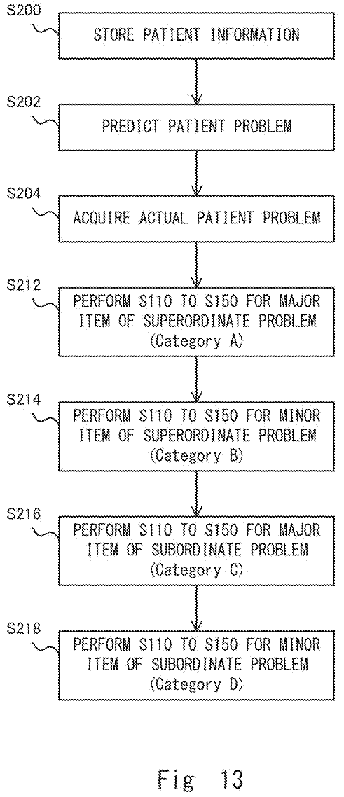

FIG. 13 is a flowchart showing a support method performed by the support apparatus 100 according to the second example embodiment. Similarly to the step S100 in FIG. 8, the patient information storage unit 110 stores patient information (Step S200). Similarly to the step S102 in FIG. 8, the patient problem prediction unit 120 predicts a patient problem (Step S202). Similarly to the step S104 in FIG. 8, the actual patient problem acquisition unit 130 acquires an actual patient problem of a subject patient (Step S204).

The support apparatus 100 performs steps S110 to S150 for the major item of the superordinate problem (i.e., for Category A) (Step S212). Specifically, the prediction matching level calculation unit 140 calculates a prediction matching level Ca for Category A (S110). That is, the prediction matching level calculation unit 140 calculates, as the prediction matching level Ca, a matching level between an item included in Category A of the actual patient problem and an item included in Category A of the predicted patient problem. Further, the alert output determination unit 150 determines whether or not the prediction matching level Ca is lower than a threshold Th1 for Category A (S120). Further, when it is determined that an alert should be output for Category A (S124), the alert type determination unit 160 determines the type of the alert for Category A (S130). The past comparison unit 162 determines the type of the alert according to the comparison between the item included in Category A of the actual patient problem and the item included in Category A of the past actual patient problem. Further, the target comparison unit 164 determines the type of the alert according to the comparison between a character string of an item included in Category A of the actual patient problem and a character string of a word included in the target. Then, the alert output unit 170 performs control so as to output the alert for Category A (S150). As described below, the same processes are performed for each of Categories B, C and D.

The support apparatus 100 performs the steps S110 to S150 for the minor item of the superordinate problem (i.e., for Category B) (Step S214). Specifically, the prediction matching level calculation unit 140 calculates a prediction matching level Ca for Category B (S110). Further, the alert output determination unit 150 determines whether or not the prediction matching level Ca is lower than the threshold Th1 for Category B (S120). Further, when it is determined that an alert should be output for Category B (S124), the alert type determination unit 160 determines the type of the alert for Category B (S130). Then, the alert output unit 170 performs control so as to output the alert for Category B (S150).

The support apparatus 100 performs the steps S110 to S150 for the major item of the subordinate problem (i.e., for Category C) (Step S216). Specifically, the prediction matching level calculation unit 140 calculates a prediction matching level Ca for Category C (S110). Further, the alert output determination unit 150 determines whether or not the prediction matching level Ca is lower than the threshold Th1 for Category C (S120). Further, when it is determined that an alert should be output for Category C (S124), the alert type determination unit 160 determines the type of the alert for Category C (S130). Then, the alert output unit 170 performs control so as to output the alert for Category C (S150).

The support apparatus 100 performs the steps S110 to S150 for the minor item of the subordinate problem (i.e., for Category D) (Step S218). Specifically, the prediction matching level calculation unit 140 calculates a prediction matching level Ca for Category D (S110). Further, the alert output determination unit 150 determines whether or not the prediction matching level Ca is lower than the threshold Th1 for Category D (S120). Further, when it is determined that an alert should be output for Category D (S124), the alert type determination unit 160 determines the type of the alert for Category D (S130). Then, the alert output unit 170 performs control so as to output the alert for Category D (S150).

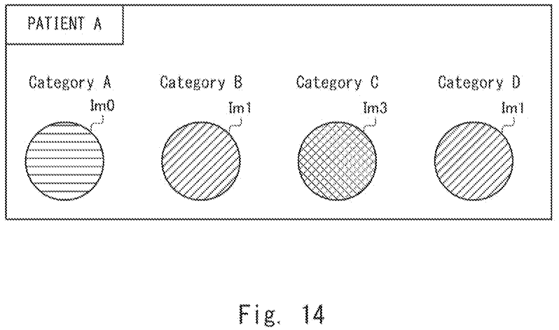

FIG. 14 shows examples of alerts that are output under control of the alert output unit 170 according to the second example embodiment. Similarly to FIG. 12, FIG. 14 shows an example in which alerts are displayed in colors. Further, similarly to FIG. 12, in the example shown in FIG. 14, an alert is displayed in a different color according to the type of the alert.

In the example shown in FIG. 14, an alert is displayed, for a patient A, for each of Categories A, B, C and D. In the example shown in FIG. 14, an alert image Im0 indicating “No alert” is displayed for Category A. Further, an alert image Im1 showing an alert of “Case A1 and Case B1” is displayed for Category B. Further, an alert image Im3 showing an alert of “Case A2 and Case B1” is displayed for Category C. Further, an alert image Im1 showing an alert of “Case A1 and Case B1” is displayed for Category D.

The support apparatus 100 according to the second example embodiment is configured so as to output an alert for each of a plurality of categories of the patient problem. In this way, it is possible to check which of a plurality of categories of the patient problem an item that is not appropriately set is included in. Therefore, it is possible to check whether or not the patient problem is appropriately set in a more detailed manner. That is, it is possible to check whether or not the patient problem is appropriately set more efficiently and more effectively.

Note that although it is assumed that an alert is output for each of all the categories of the patient problem in the example shown in FIG. 14, the configuration according to the present disclosure is not limited to such a configuration. That is, it is not necessary to output an alert for each of all the categories of the patient problem. In other words, an alert may be output for only some combinations of two of more of a plurality of categories of the patient problem. For example, an alert may be output only for Categories A and C. In this case, the processes in the steps S214 and S218 in FIG. 13 may be omitted. Further, for example, an alert may be output for Categories A, B and C. In this case, the process in the step S218 in FIG. 13 may be omitted.

### Modified Example

Note that the present invention is not limited to the above-described example embodiments, and they can be modified as appropriate without departing from the scope and spirit of the invention. For example, in each of the above-described flowcharts, the order of processes (steps) can be changed as appropriate. Further, at least one of a plurality of processes (steps) may be omitted (or skipped). For example, the process in the step S130 in FIG. 8 may be omitted. That is, it is not necessary to determine the type of an alert in this example embodiment.

Further, although the support apparatus 100 according to the above-described example embodiment supports the setting of a patient problem with which the target of rehabilitation can be achieved, the activities to which the example embodiment is applied are not limited to rehabilitation. The example embodiment can be applied to any activities aimed at improving abilities. For example, the example embodiment can be applied to habilitation. Further, for example, the example embodiment can also be applied to activities for improving sport abilities.

Further, the items included in the patient problem are classified into the superordinate problem and the subordinate problem, and are further classified into the major item and the minor item in the above-described example embodiment, the configuration according to the present disclosure is not limited to such a configuration. The items included in the patient problem may not be classified into the superordinate problem and the subordinate problem. Similarly, the items included in the patient problem may not be classified into the major item and the minor item. Further, in the above-described first example embodiment, the items included in the patient problem may not be classified into a plurality of categories. Further, in the second example embodiment, it is sufficient if the items included in the patient problem are classified into a plurality of categories, so the items included in the patient problem need not be classified into the superordinate problem and the subordinate problem, or into the major item and the minor item.

Further, the items in the patient problem that are compared when a prediction matching level is calculated may be those that are included in either of the major and minor items. In this case, in the example shown in FIG. 6, the actual patient problem may include, as items in the superordinate problem, “Walking-Right side”, “Walking-Paralyzed side”, and “IADL-Housework”. Further, the actual patient problem shown in FIG. 6 may include, as items in the subordinate problem, “Balance-Standing position”, “Balance-Sitting position”, and “Upper limb function”. Alternatively, the items in the patient problem that are compared when a prediction matching level is calculated may be those that are divided into major and minor items. In this case, in the example shown in FIG. 6, the actual patient problem may include, as items in the superordinate problem, “Walking”, “IADL”, “Right side”, “Paralyzed side”, and “Housework”. Further, the actual patient problem shown as an example in FIG. 6 may include, as items in the subordinate problem, “Balance”, “Upper limb function”, “Standing position”, and “Sitting position”. The same applies to the predicted patient problem. Further, the same applies to the calculation of a past matching level.

Further, the prediction matching level may be the number of items that are included in the actual patient problem and also included in the predicted patient problem. That is, the prediction matching level may be the number of elements in the intersection set of a set of items included in the actual patient problem and a set of items included in the predicted patient problem. The same applies to the past matching level and to the target matching level. That is, the past matching level may be the number of items that are included in the actual patient problem and also included in the past actual patient problem. Further, the target matching level may be the number of items which are included in the actual patient problem and of which character strings matches or are similar to character strings of words included in the target.

Further, the prediction matching level may be the degree of matching between the predicted patient problem and the actual patient problem. In this case, in the above-described first example of the method for calculating a prediction matching level, the prediction matching level may be the ratio of the number of items that are included in the predicted patient problem and also included in the actual patient problem to the total number of items included in the predicted patient problem. The same applies to the second example. Note that when the prediction matching level is calculated as described above, there is a possibility that the prediction matching level becomes small when the number of items in the predicted patient problem is larger than the number of items in the actual patient problem even when a lot of items in the actual patient problem are also included in the predicted patient problem as in the example shown in FIG. 6. That is, there is a possibility that the prediction matching level becomes small when the number of items in the predicted patient problem is larger than the number of items in the actual patient problem, and there are items that are not included in the actual patient problem but are included in the predicted patient problem. Meanwhile, there is a possibility that items that are not included in the actual patient problem but are included in the predicted patient problem are dealt with in the period Tc shown in FIG. 5. Therefore, it should be noted that the fact that the prediction matching level calculated as described above is small does not necessarily mean that the patient problem is not appropriately set. In other words, by using the ratio of the number of items that are in common in both the actual patient problem and the predicted patient problem to the number of items in the actual patient problem as the prediction matching level as in the first and second examples, it is possible to evaluate whether a patient problem that has been actually dealt with is appropriate or not more effectively.

The above-described program includes a set of instructions (or software codes) that, when read into a computer, causes the computer to perform one or more of the functions described in the example embodiments. The program may be stored in a non-transitory computer readable medium or in a physical storage medium. By way of example rather than limitation, a computer readable medium or a physical storage medium may include a random-access memory (RAM), a read-only memory (ROM), a flash memory, a solid-state drive (SSD), or other memory technology, a CD-ROM, a digital versatile disk (DVD), a Blu-ray (Registered Trademark) disk or other optical disk storages, a magnetic cassette, magnetic tape, and a magnetic disk storage or other magnetic storage devices. The program may be transmitted on a transitory computer readable medium or a communication medium. By way of example rather than limitation, the transitory computer readable medium or the communication medium may include electrical, optical, acoustic, or other forms of propagating signals.

Further, the whole or part of the example embodiments disclosed above can be described as, but not limited to, the following supplementary notes.

### (Supplementary Note 1)

A support apparatus comprising:
prediction means for predicting a patient problem to be dealt with in order to achieve a target of the patient;
calculation means for calculating a matching level between an actual patient problem and a predicted patient problem, the actual patient problem being a problem of the patient that has been actually dealt with for the patient, and the predicted patient problem being a predicted problem of the patient; and
output means for performing control so that an alert is output when the matching level is lower than a predetermined first threshold.

### (Supplementary Note 2)

The support apparatus described in Supplementary note 1, wherein
the prediction means calculates the predicted patient problem in a predetermined first period starting at a first time point, and
the calculation means calculates a matching level between the actual patient problem actually dealt with in a predetermined second period starting at the first time point and the predicted patient problem, the second period being shorter than the first period.
   (Supplementary note 3)

The support apparatus described in Supplementary note 2, wherein the calculation means calculates, as the matching level, a ratio of the number of items that are included in the actual patient problem and also included in the predicted patient problem to the total number of items included in the actual patient problem.

(Supplementary note 4)

The support apparatus described in Supplementary note 2, wherein the calculation means calculates, as the matching level, a ratio of the number of first items included in the predicted patient problem to the total number of the first items, the first items being items which are included in the actual patient problem and each of which a frequency at which the item has been dealt with in the second period is equal to or higher than a predetermined second threshold.

(Supplementary note 5)

The support apparatus described in any one of Supplementary notes 1 to 4, wherein
items included in the patient problem are classified into a plurality of predetermined categories,
the calculation means calculates the matching level for each of the plurality of categories, and
the output means performs control so as to output the alert for each of the plurality of categories.

### (Supplementary Notes 6)

The support apparatus described in any one of Supplementary notes 1 to 5, further comprising type determination means for determining a type of the alert based on the actual patient problem when the matching level is lower than the first threshold, wherein
the output means performs control so that the alert is output in a different form according to the determined type of the alert.

### (Supplementary Notes 7)

The support apparatus described in Supplementary note 6, wherein the type determination means determines the type of the alert according to a comparison between the actual patient problem and a patient problem actually dealt with in a period earlier than a period in which the actual patient problem has been dealt with.

### (Supplementary Notes 8)

The support apparatus described in Supplementary note 6 or 7, wherein the type determination means determines the type of the alert according to a comparison between a character string of an item included in the actual patient problem and a character string of a word included in the target.

### (Supplementary Notes 9)

A support method comprising:
predicting a patient problem to be dealt with in order to achieve a target of the patient;
calculating a matching level between an actual patient problem and a predicted patient problem, the actual patient problem being a problem of the patient that has been actually dealt with for the patient, and the predicted patient problem being a predicted problem of the patient; and
performing control so that an alert is output when the matching level is lower than a predetermined first threshold.

### (Supplementary Note 10)

The support method described in Supplementary note 9, further comprising:
calculating the predicted patient problem in a predetermined first period starting at a first time point, and
calculating a matching level between the actual patient problem actually dealt with in a predetermined second period starting at the first time point and the predicted patient problem, the second period being shorter than the first period.

### (Supplementary Note 11)

The support method described in Supplementary note 10, wherein a ratio of the number of items that are included in the actual patient problem and also included in the predicted patient problem to the total number of items included in the actual patient problem is calculated as the matching level.

### (Supplementary Note 12)

The support method described in Supplementary note 10, wherein a ratio of the number of first items included in the predicted patient problem to the total number of the first items is calculated as the matching level, the first items being items which are included in the actual patient problem and each of which a frequency at which the item has been dealt with in the second period is equal to or higher than a predetermined second threshold.

### (Supplementary Note 13)

The support method described in any one of Supplementary notes 9 to 12, wherein
items included in the patient problem are classified into a plurality of predetermined categories,
the matching level is calculated for each of the plurality of categories, and
control is performed so as to output the alert for each of the plurality of categories.

### (Supplementary Notes 14)

The support method described in any one of Supplementary notes 9 to 13, further comprising:
determining a type of the alert based on the actual patient problem when the matching level is lower than the first threshold, and
performing control so that the alert is output in a different form according to the determined type of the alert.

### (Supplementary Notes 15)

The support method described in Supplementary note 14, wherein the type of the alert is determined according to a comparison between the actual patient problem and a patient problem actually dealt with in a period earlier than a period in which the actual patient problem has been dealt with.

### (Supplementary Notes 16)

The support method described in Supplementary note 14 or 15, wherein the type of the alert is determined according to a comparison between a character string of an item included in the actual patient problem and a character string of a word included in the target.

### (Supplementary Notes 17)

A non-transitory computer readable medium storing a program for causing a computer to perform:
a step of predicting a patient problem to be dealt with in order to achieve a target of the patient;
a step of calculating a matching level between an actual patient problem and a predicted patient problem, the actual patient problem being a problem of the patient that has been actually dealt with for the patient, and the predicted patient problem being a predicted problem of the patient; and
a step of performing control so that an alert is output when the matching level is lower than a predetermined first threshold.

Although the present invention is described above with reference to example embodiments, the present invention is not limited to the above-described example embodiments. Various modifications that can be understood by those skilled in the art can be made to the configuration and details of the present invention within the scope and spirit of the invention.

This application is based upon and claims the benefit of priority from Japanese patent application No. 2021-166976, filed on Oct. 11, 2021, the disclosure of which is incorporated herein its entirety by reference.

### REFERENCE SIGNS LIST

1 SUPPORT APPARATUS
2 PREDICTION UNIT
4 CALCULATION UNIT
6 OUTPUT UNIT
50 SUPPORT SYSTEM
52 NETWORK
60 USER TERMINAL
100 SUPPORT APPARATUS
110 PATIENT INFORMATION STORAGE UNIT
120 PATIENT PROBLEM PREDICTION UNIT
130 ACTUAL PATIENT PROBLEM ACQUISITION UNIT
140 PREDICTION MATCHING LEVEL CALCULATION UNIT
150 ALERT OUTPUT DETERMINATION UNIT
160 ALERT TYPE DETERMINATION UNIT
162 PAST COMPARISON UNIT
164 TARGET COMPARISON UNIT
170 ALERT OUTPUT UNIT

What is claimed is:

1. A support apparatus comprising:
hardware, including at least one processor and at least one memory;
prediction unit implemented at least by the hardware and configured to predict a patient problem to be dealt with in order to achieve a target of the patient;
calculation unit implemented at least by the hardware and configured to calculate a matching level between an actual patient problem and a predicted patient problem, the actual patient problem being a problem of the patient that has been actually dealt with for the patient, and the predicted patient problem being a predicted problem of the patient;
type determination unit implemented at least by the hardware and configured to determine a type of the alert based on the actual patient problem when the matching level is lower than the first threshold; and
output unit implemented at least by the hardware and configured to perform control so that an alert is output in a different from according to the determined type of the alert.

2. The support apparatus according to claim 1, wherein
the prediction unit calculates the predicted patient problem in a predetermined first period starting at a first time point, and
the calculation unit calculates a matching level between the actual patient problem actually dealt with in a predetermined second period starting at the first time point and the predicted patient problem, the second period being shorter than the first period.

3. The support apparatus according to claim 2, wherein the calculation unit calculates, as the matching level, a ratio of the number of items that are included in the actual patient problem and also included in the predicted patient problem to the total number of items included in the actual patient problem.

4. The support apparatus according to claim 2, wherein the calculation unit calculates, as the matching level, a ratio of the number of first items included in the predicted patient problem to the total number of the first items, the first items being items which are included in the actual patient problem and each of which a frequency at which the item has been dealt with in the second period is equal to or higher than a predetermined second threshold.

5. The support apparatus according to claim 1, wherein
items included in the patient problem are classified into a plurality of predetermined categories,
the calculation unit calculates the matching level for each of the plurality of categories, and
the output unit performs control so as to output the alert for each of the plurality of categories.

6. (canceled)

7. The support apparatus according to claim 1, wherein the type determination unit determines the type of the alert according to a comparison between the actual patient problem and a patient problem actually dealt with in a period earlier than a period in which the actual patient problem has been dealt with.

8. The support apparatus according to claim 1, wherein the type determination unit determines the type of the alert according to a comparison between a character string of an item included in the actual patient problem and a character string of a word included in the target.

9. A support method comprising:
predicting a patient problem to be dealt with in order to achieve a target of the patient;
calculating a matching level between an actual patient problem and a predicted patient problem, the actual patient problem being a problem of the patient that has been actually dealt with for the patient, and the predicted patient problem being a predicted problem of the patient;
determining a type of the alert based on the actual patient problem when the matching level is lower than the first threshold; and
performing control so that an alert is output in a different from according to the determined type of the alert.

10. The support method according to claim 9, further comprising:
calculating the predicted patient problem in a predetermined first period starting at a first time point, and
calculating a matching level between the actual patient problem actually dealt with in a predetermined second period starting at the first time point and the predicted patient problem, the second period being shorter than the first period.

11. The support method according to claim 10, wherein a ratio of the number of items that are included in the actual patient problem and also included in the predicted patient problem to the total number of items included in the actual patient problem is calculated as the matching level.

12. The support method according to claim 10, wherein a ratio of the number of first items included in the predicted patient problem to the total number of the first items is calculated as the matching level, the first items being items which are included in the actual patient problem and each of which a frequency at which the item has been dealt with in the second period is equal to or higher than a predetermined second threshold.

13. The support method according to claim 9, wherein
items included in the patient problem are classified into a plurality of predetermined categories,
the matching level is calculated for each of the plurality of categories, and
control is performed so as to output the alert for each of the plurality of categories.

14. (canceled)

15. The support method according to claim 9, wherein the type of the alert is determined according to a comparison between the actual patient problem and a patient problem actually dealt with in a period earlier than a period in which the actual patient problem has been dealt with.

16. The support method according to claim 9, wherein the type of the alert is determined according to a comparison between a character string of an item included in the actual patient problem and a character string of a word included in the target.

17. A non-transitory computer readable medium storing a program for causing a computer to perform:
a step of predicting a patient problem to be dealt with in order to achieve a target of the patient;
a step of calculating a matching level between an actual patient problem and a predicted patient problem, the actual patient problem being a problem of the patient that has been actually dealt with for the patient, and the predicted patient problem being a predicted problem of the patient;
a step of determining a type of the alert based on the actual patient problem when the matching level is lower than the first threshold; and
a step of performing control so that an alert is output in a different from according to the determined type of the alert.

18. The support apparatus according to claim 1, wherein the output unit performs control so that an alert is displayed by a different color according to the determined type of the alert.
